(19) United States
(12) Patent Application Publication
(10) Pub. No.: US 2006/0106401 A1
(43) Pub. Date: May 18, 2006

(54) Method of inserting a flexible lens into a lens injection device and a lens injection insert for flexible or foldable lenses

(76) Inventors: Frank Deutschmann, Zittau (DE); Andreas Meyer-Rossler, Bremervorde (DE); Benno Schollmeyer, Langenfeld (DE)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 11/194,736

(22) Filed: Aug. 1, 2005

(30) Foreign Application Priority Data
Jul. 30, 2004 (DE) DE 102004037432.5

(51) Int. Cl. A61F 9/00

(52) U.S. Cl. 606/107

(57) ABSTRACT

The invention concerns a method and a lens injection insert for inserting a flexible lens into a lens injection device which has a body with a receiving means and a lens injection insert which can be inserted into the receiving means, with a storage space for lens storage, wherein the storage space is defined by mutually movable side walls of the lens injection insert, by which the volume of the storage space is variable between a transport volume for transporting the lens in a transport position in which the lens is stored in the storage space in positively locking and/or force-locking relationship and a readiness volume for readying the lens in a readiness position in which the lens can be pushed out of the storage space for injection into an eye. Following method steps: providing the lens in the lens injection insert, wherein the lens is arranged in the transport position in the lens injection insert, the lens injection insert is preserved in a physiological preserving fluid, in particular in a physiological saline solution, in a closed transport receptacle and the storage space is at least partially flooded with the preserving fluid, opening the transport receptacle, removing the lens injection insert from the transport receptacle, moving the side walls towards each other to reduce the volume of the storage space from the transport volume to the readiness volume, whereby the lens stored in the storage space is curved or rolled up and is thus moved from the transport position into the readiness position, and inserting the lens injection insert into the receiving means of the lens injection body.

The invention concerns a method of inserting a flexible or foldable lens into a lens injection device. The invention further concerns a lens injection insert for inserting a flexible or foldable lens into a lens injection device, wherein the lens injection device has a body with a receiving means and a lens injection insert which can be inserted into the receiving means, with a storage space for storing the lens, and the storage space is defined by mutually movable side walls of the lens injection insert.

Such methods and lens injection inserts have long been known. As described for example in U.S. Pat. No. 5,860,986 A, U.S. Pat. No. 6,241,737, U.S. Pat. No. 6,174,315 B1 and U.S. Pat. No. 6,312,433 B1, a lens of that kind, in order to be prepared for injection into an eye to be treated, is usually taken from a transport receptacle on the spot by the end consumer using a pair of tweezers or the like and inserted into and positioned in a receiving means of the lens injection device or a lens injection insert which can be inserted into the receiving means. A disadvantage in that respect is that that method of inserting and positioning the lens by the end consumer is complicated and requires a high level of manual skill and experience so that it can easily happen that the lens is not properly inserted or positioned. In addition the lens can be damaged by the tweezers by improper gripping thereof or it can fall out of the tweezers.

In order to eliminate the method step in regard to insertion by the end consumer US No. 2001/0001822 A1 therefore proposes providing the lens in a disposable lens injection device in which the lens is already previously inserted at the factory. A disadvantage in that respect however is that the complication and expenditure involved with the disposal process is high and the amount which is to be kept sterile from production to operative insertion, comprising the lens and the lens injection device, is correspondingly large and expensive.

Therefore the object of the invention is to provide a method of inserting a flexible lens into a lens injection device, which makes it possible to prepare the lens with fewer problems and which is less complicated and expensive.

According to the invention that object is attained in that the method has the following steps, wherein the lens injection device has a body with a receiving means and a lens injection insert which can be inserted into the receiving means, with a storage space for storing the lens, and the storage space is defined by mutually movable side walls of the lens injection insert, by which the volume of the storage space is variable between a transport volume for transporting the lens in a transport position in which the lens is supported in the storage space in positively locking and/or force-locking relationship, and a readiness volume for readying the lens in a readiness position in which the lens can be pushed out of the storage space for injection into the eye:

providing the lens in the lens injection insert, wherein the lens is arranged in the transport position in the lens injection insert, the lens injection insert is preserved in a physiological preserving fluid, in particular in a physiological saline solution, in a closed transport receptacle and the storage space is at least partially flooded with the preserving fluid,

opening the transport receptacle,

removing the lens injection insert from the transport receptacle,

moving the side walls towards each other to reduce the volume of the storage space from the transport volume to the readiness volume, whereby the lens stored in the storage space is curved or rolled up and is thus moved from the transport position into the readiness position, and

inserting the lens injection insert into the receiving means of the lens injection body.

The fact that the lens is stored in the storage space in positively locking and/or force-locking relationship in the transport position, ensures that, in the first method step, providing the lens in the lens injection insert, the lens is already stored in the proper position for the method and does not first have to be moved into that position by an end consumer. In addition that reduces the transport volume to the lens injection insert with the stored flexible or foldable lens so that the transport receptacle in which the lens injection insert with the lens therein is usually stored can approximately correspond in size to that which is used for usual transport receptacles for flexible or foldable lenses. When the transport receptacle is opened the lens injection insert can be removed from the transport receptacle for example by means of a pair of tweezers or the like without the lens being touched directly by the tweezers and thereby possibly damaged or without it being able to drop out of the tweezers.

The movement of the side walls towards each other to reduce the volume of the storage space provides that the lens is curved or rolled up in the transport space and is thus moved into a readiness position in a proper manner for the method, in which position it can be injected into an eye to be treated by way of a nozzle or a cannula. In that respect the way in which the lens can be curved or rolled up is described in further detail hereinafter.

A further method step includes insertion of the lens injection insert into the receiving means of the lens injection device for readying the lens into the lens injection device for injecting the lens into the eye to be treated.

In a variant of the method the method step of moving the side walls towards each other to reduce the volume of the storage space from the transport volume to the readiness volume, whereby the lens in the storage space is curved or rolled up and is thus moved from the transport position into the readiness position can be carried out prior to the method step in which insertion of the lens injection insert into the receiving means of the lens injection body is effected. In that respect consideration is being given in particular to the side walls being manually moved towards each other by the end user, whereby the lens is moved into the readiness position. That would have the advantage that, prior to insertion into the lens injection device, for example visual checking in regard to the proper position of the lens in the lens injection insert can be carried out and that is not complicated in terms of apparatus technology insofar as the lens injection insert is for example transparent and permits an unimpeded view into the storage space.

In another variant of the method the two method steps referred to hereinbefore, the method step of moving the side walls towards each other and that of inserting the lens injecting insert into the receiving means of the lens injection body, can take place together. That would have the advantage that the lens is moved into the readiness position only when it is inserted into the lens injection device. In addition the side walls can be moved towards each other by a structural arrangement such as for example by an introduction device with an introduction passage which tapers in a funnel configuration for introduction of the lens injection insert to an extent defined by that arrangement. In addition it is possible to provide spacers which limit the movement of the side walls towards each other, for the readiness volume. Those spacers can be provided indirectly by the side walls butting against each other with a respectively facing side.

In a development of the method, wherein the storage space is in the form of a passage with a cylindrical readiness volume, the movable side walls form the inside walls of the passage and are connected together pivotably at a respective longitudinal side about a pivot axis in the longitudinal direction of the passage and wherein the lens bears under a prestress in the transport position against the inside walls of the passage, it can be provided that the lens slides peripherally along the insides of the side walls from the transport position into the readiness position by the pivotal movement of the side walls towards each other and is curved or rolled up. The lens is held in the transport position during transport by virtue of the fact that the lens in the transport position bears under a prestress against the inside walls of the passage. In addition, with the same conditions and geometries in respect of the lens injection insert and the lens the lens always occupies the same transport position and readiness position respectively, that is to say it is always curved or rolled up to the same degree.

In a development of the method, wherein the side walls of the lens injection insert are in the form of half-shell portions of the passage with a separation plane in the longitudinal direction of the passage and the half-shell portions bear against each other in the readiness position, it can be provided that for providing the lens in the transport position the lens is introduced into the lens injection insert in such a way that the optical axis of the lens is approximately in the separation plane. That affords an optimum position for the lens in the lens injection insert as irregularities which can impede the lens in being rolled up can possibly occur in the separation plane for example as a result of fitment inaccuracies. It is assumed that the two half-shell portions bear against each other in such a way that they form a transitionless delimitation for the passage so that it is possible for the optical axis of the lens to assume any angle relative to the separation plane as the lens can then slide without any problem over the locations at which the half-shell portions bear against each other to define the readiness volume.

In addition, prior to or with insertion of the lens injection insert into the receiving means of the lens injection device, a slip gel for reducing friction can be introduced into the storage space for the lens. That is usually effected in order to reduce the friction between the lens and the lens injection insert.

The object of the invention is further attained by the provision of a lens injection insert of the kind set forth in the opening part of this specification, in that the side walls provide for variation in the volume of the storage space between a transport volume for transporting the storable lens in a transport position in which the lens is stored in the storage space in positively locking and/or force-locking relationship and a readiness volume for readying the lens in a readiness position in which the lens can be pushed out of the storage space for injection into an eye. In that respect the storage space for holding the lens can be closed at the end, whereby it is possible for the lens to be preserved in the storage space in positively locking relationship or instead knobs can be provided on the inside surface of the side walls, over which the lens can be pushed to attain the readiness position. A preferred design configuration however is one in which the maximum cross-sectional diameter of the transport volume is smaller than the diameter of the lens which can be stored therein, wherein the lens can be applied against the side walls under a resilient prestressing. That provides that the lens is held in the transport volume in force-locking relationship in its transport position.

Preferably the storage space is in the form of a passage with a substantially cylindrical readiness volume. In that case the side walls can preferably be in the form of cylindrical half-shell portions. That therefore proposes a usual configuration for the storage space, from which the lens can be expelled by a pushing device for injection into the eye to be treated.

It is further proposed that the side walls are connected together reciprocatingly pivotably by way of a hinge joint about a pivot axis in the longitudinal direction of the passage. In that case the joint can desirably be arranged respectively directly at a longitudinal side of the side walls. Preferably the hinge joint can be in the form of a film hinge. That film hinge has in particular the advantage that it is inexpensive, it can be produced together with the side walls and it is integrally joined to the side walls. In that arrangement the film hinge can be produced for example by the removal of parts of the side walls at the location at which the film hinge is to be arranged. When the lens injection insert is of a hollow-cylindrical configuration, the film hinge can be produced by tangentially removing the cylindrical casing configuration down to a residual amount which determines the thickness of the film hinge, to close to the internal space of the hollow cylinder, which forms the chamber.

Preferably the lens injection insert is produced from plastic material in an injection molding process. If now the side walls are integrally connected together by way of the film hinge, production of the lens injection insert is particularly simple in an injection molding process.

The film hinge can be elastically expandable to achieve the transport volume or an introduction volume for introducing the storable lens into the storage space, wherein the introduction volume is larger than the transport volume. When now the lens is introduced into the storage space which has been expanded to afford the introduction volume and the introduction volume is then reduced to afford the transport volume, the lens, as already described hereinbefore, is elastically prestressed, wherein the elastic prestressing of the lens is in a condition of force equilibrium with the elastic return force which is applied to the lens by the film hinge by way of the side walls. It is however also possible to provide spacers between the side walls in order to prevent the side walls from being able to unintentionally move towards each other, beyond the transport volume. Likewise it is possible to provide for arresting the side walls in the transport position and/or the readiness position.

The lens injection insert or at least the inside surfaces of the side walls, which face towards the storage space, can preferably be produced from a friction-reducing material. In a particularly preferred feature the material comprises polytetrafluoroethylene (PTFE). The use of the friction-reducing material, in particular PTFE, means that there is no longer any need to add a further friction-reducing slip gel. By virtue of the material involved, such a lens injection insert of PTFE is preferably produced, in accordance with the present state of the art, by mechanical working of a suitable blank.

In accordance with the state of the art the lens injection device can have a nozzle with a nozzle passage which, with the insertion of the lens injection insert into the receiving means, adjoins the storage space without any transition and serves for further movement of the lens which can be stored in the storage space. In that arrangement the nozzle passage can narrow in the direction facing away from the storage space so that the lens stored in the storage space in the readiness position can be further rolled up as it passes through the nozzle passage. The nozzle itself can be integrally joined to the lens injection insert. In addition the nozzle or at least the inside surfaces of the nozzle passage can be made from a friction-reducing material, in particular polytetrafluoroethylene (PTFE).

In a further development the lens injection insert can have a fixing device for fixing thereof in the receiving means of the lens injection device. In that case the fixing device can have a laterally extending projection which, as described for example in U.S. Pat. No. 6,174,315 B1, in a provided embodiment, is latchable bayonet-like in the lens injection device.

In addition the lens injection insert can have an end face which in an intended lens expulsion direction can bear against an abutment provided in the receiving means of the lens injection insert in order thereby to transmit the forces in the expulsion direction by virtue of expulsion of the lens to the lens injection device and in order to ensure that the lens injection insert is securely held in the receiving means.

The present invention is described in greater detail by means of an embodiment with an accompanying drawing in which:

FIG. 1 is a view in longitudinal section of a lens injection insert,

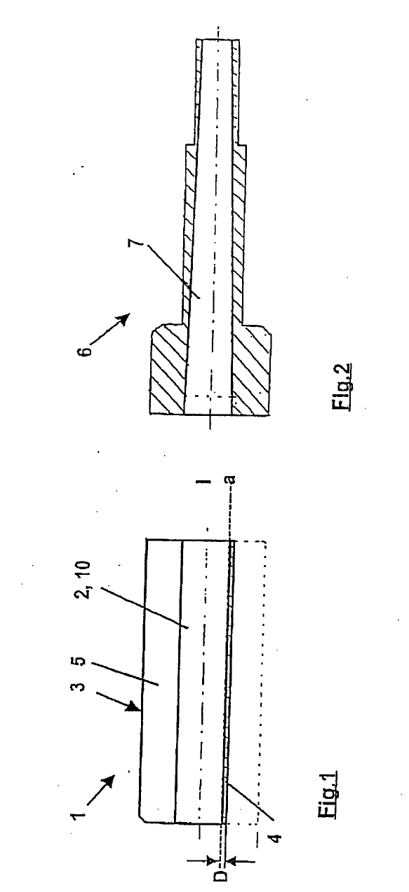

FIG. 2 is a side view of a nozzle associated with the lens injection insert,

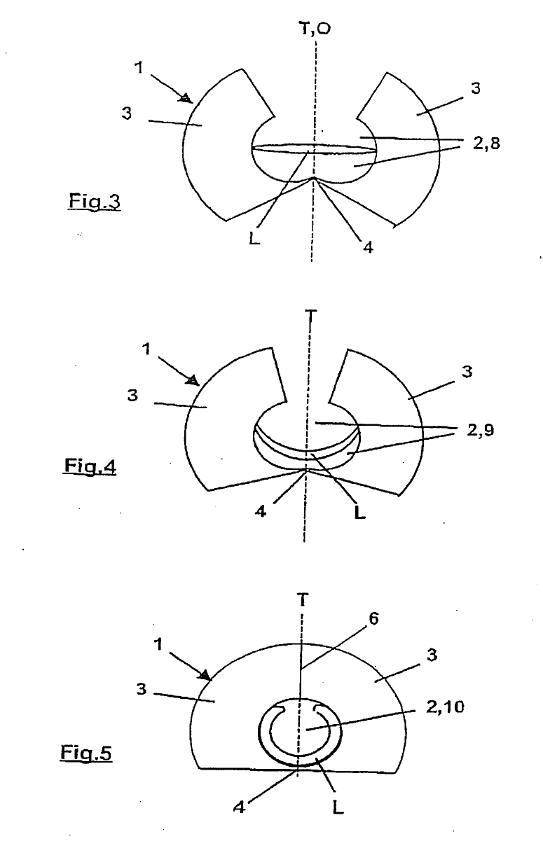

FIG. 3 is a side view of the front of the lens injection insert with a greatly expanded introduction volume together with an introduced nozzle in the introduction position,

FIG. 4 is a side view of the front of the lens injection insert with a less greatly expanded transport volume together with the introduced lens in the transport position, and

FIG. 5 is a side view showing the front of the lens injection insert in the closed condition with a readiness volume together with introduced lens in the readiness position.

FIG. 1 shows a view in longitudinal section of a lens injection insert 1 having a storage space 2 for the storage of a lens L, the storage space 2 being defined by two mutually movable side walls 3. The side walls 3 are reciprocatingly pivotably connected together by way of a film hinge 4 about a pivot axis a which extends in parallel spaced relationship with the longitudinal axis l of the lens injection insert 1. As FIGS. 3 through 5 more clearly show the two side walls 3 are of a mirror-image symmetrical configuration relative to each other, having a separation plane T as the mirror plane which in FIG. 1 is the same as the plane of the drawing. The lens injection insert 1 in this embodiment is of a hollow-cylindrical shape which, to provide the film hinge 4, was removed at one side tangentially as far as an amount which determines the thickness D of the film hinge 4 and which, to provide the two side walls 3, in the form of half-shell portions, is divided by an incision 5 which is provided at one side and which is in opposite relationship to the film hinge 4 and which extends in the longitudinal direction and which is produced radially from the outside into the storage space 2. Therefore to clearly illustrate the arrangement the original cylindrical outside contour at the tangentially removed side of the lens injection insert 1 is shown in broken line in FIG. 1.

Referring to FIG. 2, shown in a side view is a nozzle 6 which is associated with the lens injection insert 1 and which upon being inserted into a lens injection device (not shown here) bears at the end against the lens injection insert 1, the storage space 2 going without a transition into a nozzle passage 7 provided in the nozzle 6. In this embodiment the nozzle passage 7 is of a conical configuration, whereby the lens (not shown in FIGS. 1 and 2) can be further rolled up. The nozzle passage however can also be designed without a conical taper as the lens injection insert 1 can be so designed that the lens L can be rolled up in the chamber 2 to such an extent that it can be injected into an eye to be treated without being further rolled up in the nozzle passage 7.

FIGS. 3 through 5 are end views of the lens injection insert 1 with a lens L in the storage space 2, wherein the side walls 3 of the lens injection insert 1 are shown in various pivotal positions: in FIG. 3 the side walls 3 are shown in a position in which the storage space 2 is expanded to afford an introduction volume 8 and the lens L is arranged in an introduction position in the storage space 2 in such a way that it bears laterally against the side walls 3 and its optical axis o is in the separation plane T. That expansion of the storage space 2 is effected by the action of an external force against the elastic force of the film hinge 4 so that the film hinge 4 in the introduction position exerts on the side walls 3 an elastic return force which is further transmitted to the lens L by way of the side walls 3. When the external force acting on the lens injection device 1 is removed, the side walls 3 are moved towards each other about the pivot axis a by way of the elastic return force of the film hinge 4 whereby the lens L in the storage space 2 is elastically curved until there is an equilibrium in respect of the elastic return forces of the lens L and the film hinge 4. That causes a reduction in the volume of the storage space 2 from the introduction volume to a transport volume 9 while the lens L is in a transport position (FIG. 4). Due to the renewed action of an external force which however is now applied to the side walls 3 to move the side walls 3 towards each other the side walls 3 come into a condition of mutual contact as shown in FIG. 5, wherein the storage space 2 has decreased in size to a readiness volume 10 which is of a substantially cylindrical configuration while the lens L is in a readiness position in which it is more substantially curved or rolled up.

When the lens L is in the transport position, it bears under a prestressing in force-locking relationship against the inside surfaces of the side walls 3. When the side walls 3 are further moved towards each other, under the further effect of the forces involved, the lens L slides against the insides of the side walls 3 and is thereby further rolled up. In that situation the lens L can be rolled up to an extent such that the lens L does not have to be further rolled up in the nozzle passage 7 for injection into an eye to be treated (not shown), through the nozzle 6. After injection of the lens L into the eye the lens injection insert 1 can be re-used.

Advantageously the injection insert comprises a friction-reducing material, in particular polytetrafluoroethylene (PTFE). It is likewise proposed that the nozzle is also made from PTFE.

The lens injection insert 1 can be produced in the form of an injection molding. The lens injection insert 1 can be produced in one injection molding operation by virtue of its film hinge 4, by way of which the side walls 3 are integrally connected together. When using PTFE as the material, as described by way of example hereinafter, in accordance with the present state of the art, mechanical working of an appropriately suitable blank is necessary instead of production by injection molding.

### LIST OF REFERENCES

1 lens injection insert
2 storage space
3 side wall
4 film hinge
5 incision
6 nozzle
7 nozzle passage
8 introduction volume
9 transport volume
10 readiness volume
a pivot axis
l longitudinal axis
D thickness
T separation plane
L lens
o optical axis

1. A method of inserting a flexible lens into a lens injection device, wherein the lens injection device has a body with a receiving means and a lens injection insert which can be inserted into the receiving means, with a storage space for storing the lens, and the storage space is defined by mutually movable side walls of the lens injection insert, by which the volume of the storage space is variable between a transport volume for transporting the lens in a transport position in which the lens is supported in the storage space in positively locking and/or force-locking relationship, and a readiness volume for readying the lens in a readiness position in which the lens can be pushed out of the storage space for injection into the eye, comprising the following method steps:
providing the lens in the lens injection insert, wherein the lens is arranged in the transport position in the lens injection insert, the lens injection insert is preserved in a physiological preserving fluid, in particular in a physiological saline solution, in a closed transport receptacle and the storage space is at least partially flooded with the preserving fluid,
opening the transport receptacle,
removing the lens injection insert from the transport receptacle,
moving the side walls towards each other to reduce the volume of the storage space from the transport volume to the readiness volume, whereby the lens stored in the storage space is curved or rolled up and is thus moved from the transport position into the readiness position, and
inserting the lens injection insert into the receiving means of the lens injection body.

2. A method as set forth in claim 1 characterised in that the method step of moving the side walls towards each other to reduce the volume of the storage space from the transport volume to the readiness volume, whereby the lens stored in the storage space is curved or rolled up and is thus moved from the transport position into the readiness position, is effected prior to the method step in which insertion of the lens injection insert into the receiving means of the lens injection body is effected.

3. A method as set forth in claim 1 characterised in that the method step of moving the side walls towards each other to reduce the volume of the storage space from the transport volume to the readiness volume, whereby the lens stored in the storage space is curved or rolled up and is thus moved from the transport position into the readiness position, is effected together with the method step in which insertion of the lens injection insert into the receiving means of the lens injection body is effected.

4. A method as set forth in claim 1 wherein the storage space is in the form of a passage with a cylindrical readiness volume, the movable side walls form the inside walls of the passage and are respectively connected together pivotably at a longitudinal side about a pivot axis in the longitudinal direction of the passage and wherein the lens in the transport position bears against the inside walls of the passage under a prestressing, characterised in that the lens slides peripherally along the insides of the side walls from the transport position into the readiness position by virtue of the pivotal movement of the side walls towards each other, and is curved or rolled up.

5. A method as set forth in claim 4 wherein the side walls of the lens injection insert are in the form of half-shell portions of the passage with a separation plane in the longitudinal direction of the passage and the half-shell portions bear against each other in the readiness position characterised in that for providing the lens in the transport position the lens is so introduced into the lens injection insert that the optical axis of the lens is approximately in the separation plane.

6. A method as set forth in claim 1 characterised in that prior to or with insertion of the lens injection insert into the receiving means of the lens injection device a slip gel for reducing friction is introduced into the storage space.

7. A lens injection insert for inserting a flexible or foldable lens into a lens injection device, wherein the lens injection device has a body with a receiving means and a lens injection insert which can be inserted into the receiving means, with a storage space for storing the lens, and the storage space is defined by mutually movable side walls of the lens injection insert, characterised in that the side walls provide for variation in the volume of the storage space between a transport volume for transporting the storable lens in a transport position in which the lens is stored in the storage space in positively locking and/or force-locking relationship and a readiness volume for readying the lens in a readiness position in which the lens can be pushed out of the storage space for injection into an eye.

8. A lens injection insert as set forth in claim 7 characterised in that the maximum cross-sectional diameter of the transport volume is smaller than the diameter of the lens which can be stored therein, wherein the lens can bear against the side walls under an elastic prestressing.

9. A lens injection insert as set forth in claim 7 characterised in that the storage space is in the form of a passage with a substantially cylindrical readiness volume.

10. A lens injection insert as set forth in claim 8 characterised in that the side walls are reciprocatingly pivotably connected together by way of a hinge joint about a pivot axis in the longitudinal direction of the passage.
